(19) United States
(12) Patent Application Publication
(10) Pub. No.: US 2026/0311833 A1
(43) Pub. Date: Oct. 8, 2026

(54) METHOD, DEVICE, AND COMPUTER PROGRAM FOR AUTOMATICALLY GENERATING MEDICAL CHART BY USING GENERATIVE ARTIFICIAL INTELLIGENCE MODEL

(71) Applicant: Synergy A.I. Co. Ltd., Seoul (KR)

(72) Inventor: Taeyoung SHIN, Seoul (KR)

(73) Assignee: Synergy A.I. Co. Ltd., Seoul (KR)

(21) Appl. No.: 19/643,956

(22) Filed: Apr. 10, 2026

Related U.S. Application Data
(63) Continuation of application No. PCT/KR2024/012709, filed on Aug. 26, 2024.

(30) Foreign Application Priority Data
Oct. 13, 2023 (KR) 10-2023-0137015
Aug. 8, 2024 (KR) 10-2024-0106191

(51) Int. Cl. G16H 15/00; G16H 10/60

(52) U.S. Cl. CPC G16H 15/00; G16H 10/60

(57) ABSTRACT

Provided are a method, an apparatus, and a computer program for automatically generating a medical chart by using a generative artificial intelligence model. The method for automatically generating a medical chart using a generative artificial intelligence model is performed by a computing device and comprises the steps of: acquiring medical treatment data of a patient; and acquiring a medical chart corresponding to the acquired medical treatment data by using a pre-trained generative artificial intelligence (AI) model.

### CROSS-REFERENCE TO RELATED PATENT APPLICATION

This application is a Continuation of International Application No. PCT/KR2024/012709, filed on Aug. 26, 2024, which claims the benefit of Korean Patent Applications Nos. 10-2024-0106191,filed on Aug. 8, 2024, and 10-2023-0137015, filed on Oct. 13, 2023, in the Korean Intellectual Property Office, the disclosure of which is incorporated herein in its entirety by reference.

### TECHNICAL FIELD

Various embodiments of the present invention relate to a method, a device, and a computer program for automatically generating a medical chart using a generative artificial intelligence (AI) model.

### BACKGROUND ART

It is common for doctors to create medical charts for patients during the course of their medical consultations.

Here, a medical chart (or a patient chart or medical record) is a file or electronic document containing all information related to a patient's medical history. The medical chart may provide a comprehensive record of medical care that the patient has received and include various information on the patient and his or her illness. For example, the medical chart may include the patient's basic information (e.g., personal information such as the patient's name, age, gender, address, etc.), medical history (e.g., information on past and current medical records, surgical history, allergies, medications currently being taken, etc.), details of medical care (e.g., documents recording the details of each visit to a clinic or hospital, test results, medical opinions, etc.), diagnostic information (e.g., information such as current and past diagnoses, a medical history, etc.), test result information (e.g., results of various tests such as a blood test, an X-ray, magnetic resonance imaging (MRI), etc.), treatment and prescription information (e.g., treatments provided to the patient, prescribed medications, and the patient's response thereto), consultation records (e.g., the content of consultations or discussions between a doctor and the patient), vaccination information (e.g., the patient's vaccination history), comprehensive assessment information (e.g., assessments and opinions provided by various specialists (e.g., a neurologist, a psychiatrist, a cardiologist, etc.), and the like.

The subjective, objective, assessment, and plan (SOAP) medical chart (SOAP notes) is a template for creating such medical charts and provides a methodology for documenting a patient's clinical course in a specific and systematic manner. Here, SOAP is divided into four parts: a subjective(S) part where the patient's self-report of his or her condition is recorded, an objective (O) part where specific and measurable data regarding the patient's condition is recorded, an assessment (A) part where a medical worker assesses the patient's condition, and a plan (P) part where a treatment and recovery plan for the patient is recorded.

Meanwhile, medical staff conventionally record medical charts manually using input devices such as a mouse or keyboard. However, this method of creating medical charts involves a somewhat cumbersome process, requiring a significant investment of time and effort.

Further, since the conventional method of creating medical charts is performed manually in detail, there is a risk of human error such as typographic errors, the input of incorrect information, or the like.

The foregoing background art was possessed or acquired by the inventor in the course of deriving the subject matter of the present invention and is not necessarily considered prior art that was disclosed to the general public prior to the filing of the present application.

### DETAILED DESCRIPTION OF INVENTION

### Technical Problem

An object to be achieved by the present invention is providing a method, an apparatus, and a computer program for automatically generating a medical chart using a generative artificial intelligence (AI) model that process clinical data collected during a patient consultation process to automatically generate a medical chart using a preset format of a template and thereby facilitate the creation of medical charts while reducing human errors, which enables the creation of accurate medical charts.

Objects to be achieved by the present invention are not limited to that described above, and other objects that have not been described will be clearly understood by those of ordinary skill in the art from the following description.

### Technical Solution

To achieve the above-described purpose, a method of automatically generating a medical chart using a generative artificial intelligence (AI) model according to an embodiment of the present invention is performed by a computing device and includes acquiring clinical data of a patient and acquiring a medical chart corresponding to the acquired clinical data using a pretrained generative AI model.

According to various embodiments, the acquired clinical data may be data including information on a medical consultation with the patient and may be data in the form of voice generated by recording voice from a time point where a first user input is acquired from a medical worker who treats the patient to a time point where a second user input is acquired.

According to various embodiments, the acquiring of the medical chart may include generating input data on the basis of the acquired clinical data and acquiring a medical chart corresponding to the acquired clinical data as resulting data by inputting the generated input data into the pretrained generative AI model.

According to various embodiments, the acquired clinical data may include clinical data in the form of voice obtained by recording a medical consultation with the patient, and the generating of the input data may include converting the clinical data in the form of voice into clinical data in the form of text, generating a prompt including a command to indicate a type of medical chart to be acquired and generate a medical chart, and generating input data including the converted clinical data in the form of text and the generated prompt.

According to various embodiments, the acquiring of the medical chart may include preprocessing the acquired clinical data and acquiring a medical chart using the preprocessed clinical data.

According to various embodiments, the preprocessing of the acquired clinical data may include extracting information including a consultation-related keyword among a plurality of pieces of information included in the acquired clinical data as required information or filtering out information including a consultation-unrelated keyword as unrequired information.

According to various embodiments, the preprocessing of the acquired clinical data comprises setting an information filtering criterion on the basis of a type of medical chart to be acquired and extracting only necessary information among a plurality of pieces of information included in the acquired clinical data or filtering out unnecessary information on the basis of the set information filtering criterion.

According to various embodiments, the acquiring of the medical chart may include analyzing the acquired clinical data to extract a plurality of keywords, selecting any one from a plurality of preset medical chart formats on the basis of the plurality of extracted keywords, and acquiring a medical chart by processing the acquired clinical data in accordance with the selected medical chart format.

According to various embodiments, the acquiring of the medical chart may include extracting keywords corresponding to each of a plurality of items included in a preset medical chart format from the acquired clinical data, acquiring information corresponding to each of the plurality of items on the basis of the extracted keywords, and when no keyword corresponding to any one of the plurality of items is extracted, acquiring information corresponding to the item on the basis of keywords extracted in accordance with the items other than the item.

According to various embodiments, the preset medical chart format may be a subjective, objective, assessment, and plan (SOAP) medical chart format including a subjective item where information on a patient's complaint is recorded, an objective item where factual information collected during a consultation process is recorded by a medical worker, an assessment item where assessment information generated by a medical worker assessing the patient's current condition is recorded, and a plan item where treatment and follow-up plan information for the patient is recorded.

According to various embodiments, the method may further include providing a user interface (UI) configured to output the acquired clinical data and the acquired medical chart, and the provided UI may include a first region where the acquired clinical data is output and a second region where the acquired medical chart is output.

According to various embodiments, the providing of the UI may include, when a plurality of different kinds of medical charts are acquired in accordance with the acquired clinical data, separately outputting the plurality of acquired medical charts in different regions of the provided UI.

To achieve the above-described purpose, a computing device for performing a method of automatically generating a medical chart using a generative AI model according to another embodiment of the present invention includes a processor, a network interface, a memory, and a computer program loaded into the memory and executed by the processor. The computer program includes an instruction to acquire clinical data of a patient and an instruction to acquire a medical chart corresponding to the acquired clinical data using a pretrained generative AI model.

To achieve the above-described purpose, a computer program according to still another embodiment of the present invention is recorded on a recording medium readable by a computing device to perform, in combination with a computing device, a method of automatically generating a medical chart using a generative AI model, the method including acquiring clinical data of a patient and acquiring a medical chart corresponding to the acquired clinical data using a pretrained generative AI model.

Other details of the present invention are included in the detailed description and drawings.

### Advantageous Effects

An object to be achieved by the present invention is to automatically generate a medical chart on the basis of a generative artificial intelligence (AI) model by processing clinical data collected during a patient consultation process to automatically generate a medical chart in a preset format, thereby facilitating the creation of medical charts and also reducing human errors, which enables the creation of accurate medical charts.

Effects of the present invention are not limited to that described above, and other effects that have not been described will be clearly understood by those of ordinary skill in the art from the following description.

### BRIEF DESCRIPTION OF DRAWINGS

The following drawings appended to the present specification illustrate exemplary embodiments of the present invention and help with understanding of the technical spirit of the present invention in conjunction with the detailed description of the present invention. Therefore, the present invention should not be interpreted as being limited to only matters described in the drawings.

FIG. 1 is a diagram showing a system for automatically generating a medical chart using a generative artificial intelligence (AI) model according to an embodiment of the present invention.

FIG. 2 is a hardware configuration diagram of a computing device according to another embodiment of the present invention.

FIG. 3 is a flowchart illustrating a method of automatically generating a medical chart using a generative AI model according to still another embodiment of the present invention.

FIG. 4 is a flowchart illustrating a method of acquiring a medical chart through a generative AI model according to various embodiments.

FIGS. 5 to 9 are diagrams illustrating user interfaces (UIs) applicable to various embodiments.

### MODES OF THE INVENTION

The advantages and features of the present invention and a method of achieving them will become apparent from embodiments which will be described in detail below with reference to the accompanying drawings. However, the present invention is not limited to the embodiments disclosed below and may be implemented in various different forms. The embodiments are only provided to make the disclosure of the present invention complete and fully convey the scope of the present invention to those skilled in the technical field to which the present invention pertains. The present invention is only defined by the scope of the claims.

Terminology used in this specification is only for the purpose of describing embodiments and is not intended to limit the present invention. In this specification, singular forms include plural forms as well unless the context particularly indicates otherwise. The terms “comprises” and/or “comprising” used in this specification do not preclude the presence or addition of one or more components other than stated components.

Throughout the specification, like reference numerals refer to like components, and the term “and/or” includes any of stated components or any combination thereof. Although the terms “first,” “second,” etc., are used to describe various components, these components are not limited by the terms. These terms are used for the sole purpose of distinguishing one component from others. Therefore, a first component mentioned below may be a second component within the technical scope of the present invention.

As used herein, the term “unit” or “module” refers to a software component or a hardware component such as a field programmable gate array (FPGA) or an application-specific integrated circuit (ASIC), and a “unit” or “module” performs certain roles. However, a “unit” or “module” is not limited to software or hardware. A “unit” or “module” may be configured to reside in an addressable storage medium or reproduce one or more processors. Accordingly, as an example, a “unit” or “module” includes components, such as software components, object-oriented software components, class components, and task components, processes, functions, attributes, procedures, subroutines, segments of program code, drivers, firmware, microcode, circuitry, data, databases, data structures, tables, arrays, and variables. Components and functions provided in “units” or “modules” may be combined into a smaller number of components, and “units” or “modules” may be subdivided into additional components and “units” or “modules.”

Spatially relative terms such as “below,” “beneath,” “lower,” “above,” “upper,” etc., may be used for easily describing the relationship between one component and other components as shown in the drawings. It is to be understood that the spatially relative terms are intended to encompass different orientations of components upon their use or operation in addition to the orientation depicted in the drawings. For example, when a component shown in a drawing is turned over, components described as “below” or “beneath” another component may then be oriented “above” the other component. Therefore, the exemplary term “below” may encompass orientations of both above and below. A component may be otherwise oriented, and the spatially relative terms may be interpreted accordingly.

Expressions such as “first,” “second,” etc., are used herein to distinguish one target from another when referring to a plurality of targets of the same kind, unless the context describes otherwise. The expressions do not imply any order or importance of the targets.

The expressions “A, B, and C,” “A, B, or C,” “A, B, and/or C,” “at least one of A, B, and C,” “at least one of A, B, or C,” “at least one of A, B, and/or C,” “at least one selected from A, B, and C,” “at least one selected from A, B, or C,” “at least one selected from A, B, and/or C,” etc., used herein may indicate any of listed components or all possible combinations thereof. For example, “at least one selected from A and B” may indicate (1) A, (2) at least one of A, (3) B, (4) at least one of B, (5) at least one of A and at least one of B, (6) B and at least one of A, (7) A and at least one of B, and (8) both A and B.

The expression “on the basis of” is used herein to describe one or more factors that affect the action or operation of determination or judgment described in a phrase or sentence including the expression. This expression does not exclude additional factors that affect the action or operation of the determination or judgment.

When a component (e.g., a first component) is referred to as being “connected” or “coupled” to another component (e.g., a second component), the component may be directly connected or coupled to the other component or indirectly connected or coupled to the other component via a new component (e.g., a third component).

As used herein, the expression “configured to” may have a meaning such as “set to,” “capable of,” “changed to,” “made to,” “able to,” etc., depending on the context. The expression is not limited to the meaning “specially designed as hardware.” For example, a processor configured to perform a specific operation may be a generic-purpose processor that may perform the specific operation by executing software.

Unless defined otherwise, all terms used herein (including technical or scientific terms) have the same meanings as those generally understood by those skilled in the technical field to which the present invention pertains. In addition, terms defined in commonly used dictionaries are not interpreted ideally or excessively unless clearly and particularly defined herein.

In this specification, a computer is any type of hardware device including at least one processor and may be understood as collectively including a software element operating in the corresponding hardware device according to embodiments. For example, a computer may be understood with, but is not limited to, a meaning including all of a smartphone, a tablet personal computer (PC), a desktop computer, a notebook computer, and a user client and an application running on each device.

Hereinafter, embodiments of the present invention will be described in detail with reference to the accompanying drawings.

Although each step described in this specification is described as being performed by a computer, the subject of each step is not limited thereto, and at least some steps may be performed in different devices according to embodiments.

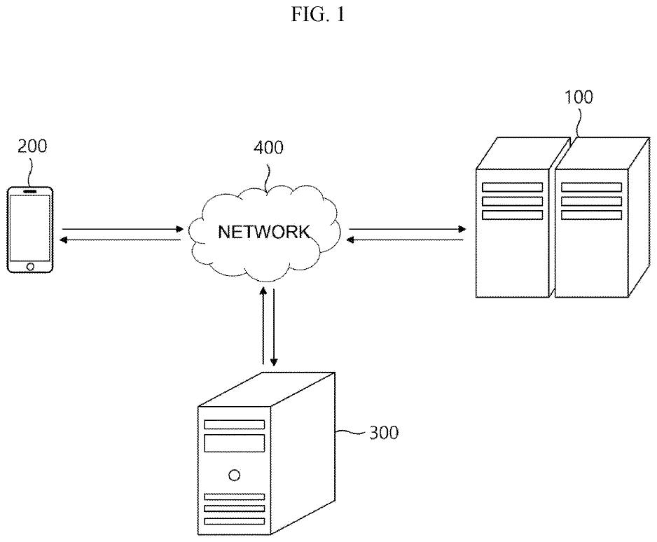

FIG. 1 is a diagram showing a system for automatically generating a medical chart using a generative artificial intelligence (AI) model according to an embodiment of the present invention.

Referring to FIG. 1, the system for automatically generating a medical chart using a generative AI model according to the embodiment of the present invention may include a computing device 100, a user terminal 200, an external server 300, and a network 400.

Here, the system for automatically generating a medical chart using a generative AI model shown in FIG. 1 is in accordance with the embodiment, and components of the system are not limited to the embodiment shown in FIG. 1 and may be added, changed, or removed as necessary.

According to the embodiment, the computing device 100 may perform an automatic medical chart generation process.

According to various embodiments, the computing device 100 may acquire clinical data including information on a medical consultation with a patient from a medical worker and acquire a medical chart corresponding the clinical data using a pretrained AI model.

The AI model (e.g., a neural network) is composed of one or more network functions, and the one or more network functions may generally be a set of interconnected calculation units that may be referred to as “nodes.” These “nodes” may also be referred as “neurons.” The one or more network functions include one or more nodes. Nodes (or neurons) constituting one or more network functions may be interconnected via one or more “links.”

In the AI model, one or more nodes connected via a link may have a relative relationship of an input node and an output node. An input node and an output node are relative concepts. Any node which is an output node with respect to one node may be an input node with respect to another node, and vice versa. As described above, the relationship of an input node and an output node may be established on the basis of a link. One or more nodes may be connected to one input node, and vice versa.

In the relationship of an input node and an output node connected via one link, a value of the output node may be determined on the basis of data input to the input node. Here, the node connecting the input node and the output node to each other may have a weight. The weight may be variable and may be varied by a user or an algorithm to perform a function wanted by the AI model. For example, when one or more input nodes are connected to one output node via separate links thereof, the output node may determine an output node value on the basis of values input to the input nodes connected to the output node and weights set for the links corresponding to each of the input nodes.

As described above, in the AI model, one or more nodes are connected to each other via one or more links and have the relationship of an input node and an output node. A characteristic of the AI model may be determined in accordance with the number of nodes and links in the AI model, connections between the nodes and the links, and weights assigned to the links. For example, when there are two AI models in which the same numbers of nodes and links are present and the corresponding links have different weights, the two AI models may be considered to be different.

Some nodes constituting the AI node may constitute one layer on the basis of distances from an initial input node. For example, a set of nodes having a distance of n from the initial node may constitute a layer n. The distance from the initial input node may be defined by the minimum number of links to be passed through to reach a corresponding node from the initial input node. However, the definition of a layer is arbitrary for description, and the order of a layer in the AI model may be defined in a different way from that described above. For example, a layer of nodes may be defined by a distance from a final output node.

The initial input node may be one or more nodes to which data is directly input without passing through a link in the relationship with other nodes among the nodes in the AI model. Alternatively, the initial input node may be a node that does not have other input nodes connected via links in the relationship between nodes based on links in the AI model network. Similarly, the final output node may be one or more nodes that do not have an output node in the relationship with other nodes among the nodes in the AI model. Also, a hidden node may be a node constituting the AI model other than the input node and the final output node. The AI model according to the embodiment of the present invention may have a form where the number of nodes in an input layer is larger than the number of nodes in a hidden layer close to the output layer and the number of nodes decreases from the input layer to the hidden layer.

The AI model may include one or more hidden layers. Hidden nodes in the hidden layers may use an output of a previous layer and outputs of surrounding hidden nodes as inputs. The hidden layers may have the same or different numbers of hidden nodes. The number of nodes in the input layer may be determined on the basis of the number of data fields of input data and may be the same as or different from the number of hidden nodes. The input data input to the input layer may be computed by the hidden nodes of the hidden layers, and a computation result may be output by a fully connected layer (FCL) which is the output layer.

In various embodiments, the AI model may be a deep learning model.

The deep learning model (e.g., a deep neural network (DNN)) may be an AI model including a plurality of hidden layers in addition to an input layer and an output layer. When the DNN is used, latent structures of data may be determined. In other words, latent structures of photos, text, videos, voice, and music (e.g., what objects are in the photos, what the content and feelings of the text are, what the content and feelings of the voice are, etc.) may be determined.

The DNN may be, but is not limited to, a convolutional neural network (CNN), a recurrent neural network (RNN), an autoencoder, a generative adversarial network (GAN), a restricted Boltzmann machine (RBM), a deep belief network (DBN), a Q network, a U network, a Siamese network, and the like.

In various embodiments, the network functions may include an autoencoder. Here, the autoencoder may be an artificial neural network for outputting output data similar to input data.

The autoencoder may include at least one hidden layer, and an odd number of hidden layers may be disposed between input and output layers. The number of nodes in each layer may decrease from the input layer to an intermediate layer called a bottleneck layer (encoding) and then increase from the bottleneck layer to the output layer (symmetrical to the input layer) symmetrically to the decrease. The number of nodes of a dimension reduction layer and the number of nodes of a dimension restoration layer may or may not be symmetrical. In addition, the autoencoder may perform non-linear dimension reduction. The number of input and output layers may correspond to the number of sensors remaining after preprocessing of input data. The autoencoder may have a structure in which the number of nodes in a hidden layer included in the encoder decreases with an increase in the distance from the input layer. When the number of nodes in the bottleneck layer (a layer having the smallest number of nodes positioned between the encoder and the decoder) is too small, a sufficient amount of information may not be delivered, and thus the number of nodes in the bottleneck layer may be maintained at a specific number or more (e.g., half the number of nodes in the input layer or more).

In various embodiments, the AI model may be a generative AI model.

The generative AI model may be a natural language model that has been pretrained on large-scale data using a transformer. A transformer model is based on the attention mechanism. In addition to having an encoder-decoder architecture, the transformer model addresses the drawbacks of existing sequence-to-sequence models and is characterized by the design of an encoder-decoder that uses no RNN.

While attention has been used to complement RNNs of sequence-to-sequence models, an encoder-decoder has been designed in transformers using only attention without RNNs. An attention technique used in transformers is referred to as “multi-head attention” or “self-attention.”

When an input (an input sequence) of the encoder and an input (a word constituting an output sequence) of the decoder are given, a transformer model is trained to increase the probability of a correct answer (the next word of the output sequence).

A pretrained language model may be a language model that has been trained to perform a specific task using large-scale language data (corpus). For example, GPT models learn a task of predicting the next word given a sequence of preceding words and update themselves to correctly predict the next word, thereby performing a learning process. As another example, bidirectional encoder representations from transformers (BERT) models learn a task of predicting a missing word between given words on either side and update themselves to correctly predict the missing word, thereby performing a learning process.

Even when large-scale language data which may be relatively easy to collect is input without additional preprocessing, this task enables a computer to perform self-supervised learning by covering and guessing words one by one on its own. When a model that has been pretrained on large-scale language data in this way is completed, the model may be fine-tuned and utilized to perform various detailed tasks.

Fine-tuning is a transfer learning method in which a model trained on a specific task is retrained to perform another task and is a method of relatively easily training a pretrained model on a new task by utilizing the experience of the pretrained model.

For example, a pretrained language model may have been trained to guess a missing word, but a model for generating an answer to a question or classifying a document may be obtained by fine-tuning the pretrained language model.

By significantly scaling up these pretrained language models and training them on an enormous amount of data, results that far surpass the performance of existing AI models are obtained. AI models developed in this way are referred to as hyperscale AI or large language models (LLMs).

Here, a method of automatically generating a medical chart using a generative AI model according to various embodiments of the present invention may be implemented when one AI model is built and used by multiple medical workers to acquire medical charts. In some cases, AI models corresponding to each of multiple medical workers, that is, AI models customized to medical workers, may be built, and each medical worker may utilize his or her own customized AI model to acquire a medical chart.

For example, the computing device 100 may build an AI model customized to a first medical worker using, as training data, clinical data of the first medical worker and medical charts made by the first medical worker, and when clinical data is acquired from the first medical worker, may generate a medical chart corresponding to the clinical data through the AI model customized to the first medical worker.

Also, the computing device 100 may build an AI model customized to a second medical worker using, as training data, clinical data of the second medical worker and medical charts made by the second medical worker, and when clinical data is acquired from the second medical worker, may generate a medical chart corresponding to the clinical data through the AI model customized to the second medical worker. In this way, by building and utilizing an AI model customized to each medical worker to generate a medical chart, it is possible to create a medical chart in a format and style preferred by the medical worker.

In some cases, when the second medical worker requests the generation of a medical chart in a format and style preferred by the first medical worker, the computing device 100 may input clinical data acquired from the second medical worker to the AI model customized to the first medical worker, thereby generating a medical chart corresponding to the clinical data through the AI model customized to the first medical worker.

In various embodiments, the computing device 100 may generate a medical chart corresponding to clinical data through the AI model, but the medical chart may be acquired in a language preset by a medical worker. To this end, the AI model may be implemented to support various languages when trained on training data in the various languages.

Here, the method of automatically generating a medical chart using a generative AI model according to various embodiments of the present invention has been described as automatically acquiring a medical chart through an AI model but is not limited thereto. The method may be implemented to provide a comprehensive medical solution such as utilizing an AI model to diagnose and predict a patient's condition and develop a clinical plan accordingly.

In various embodiments, the computing device 100 may acquire symptom data including the patient's symptom details from the patient, acquire medical solution information corresponding to the symptom data using the pretrained AI model, and provide the acquired medical solution information to the patient.

For example, when the symptom data including the patient's symptom details (e.g., symptom data generated in the form of voice by recording the symptom details) is acquired from the patient, the computing device 100 may analyze the symptom data to make a diagnosis for the patient and may derive treatment and prescription information in accordance with the diagnosis results. Also, the computing device 100 may generate medical solution information including the diagnosis results and the derived treatment and prescription information and provide the medical solution information to the patient.

When the computing device 100 receives image data uploaded by the patient along with the symptom data, the computing device 100 may analyze the image data on the basis of the symptom details included in the symptom data, and based on the analysis, generate and provide medical solution information including diagnostic, treatment, and prescription information for the patient to the patient.

As an example, the computing device 100, having acquired an X-ray along with the symptom data from the patient, may analyze the X-ray to determine the condition of the lungs, heart, bones, etc., when a symptom detail included in the symptom data is “chest pain.” Then, the computing device 100 may analyze the cause of the “chest pain,” diagnose the patient in accordance with the analyzed cause, and derive treatment and prescription information on the basis of the diagnosis result. Also, the computing device 100 may generate and provide medical solution information including the derived treatment and prescription information to the patient.

To this end, the AI model may be a multimodal model that receives two or more different kinds of data and outputs one piece of result data, but is not limited thereto.

In various embodiments, when treatment image data including treatment details is acquired from a medical worker and/or the patient, the computing device 100 may acquire treatment guidance information by analyzing the treatment image data through the AI model.

For example, when video data related to a treatment action is acquired from the medical worker and/or the patient, the computing device 100 may extract treatment-related information by analyzing each of a plurality of frames included in the acquired video data and acquire treatment guidance information including the extracted information.

Here, the treatment-related information included in the treatment guidance information may include, but is not limited to, a treatment area, a frame position corresponding to a time point where the treatment is performed, a frame position corresponding to a time point where the treatment is finished, and information on treatment actions performed on the treatment area.

Here, the method of automatically generating a medical chart using a generative AI model according to various embodiments of the present invention has been described as providing an in-visit service based on a generative AI model in which a medical chart is automatically acquired using clinical data generated when a patient visits a medical worker and receives an in-person medical consultation. However, the method is not limited thereto, and a pre-visit service and a post-visit service may also be provided.

First, the pre-visit service may represent a service for providing a brief pre-visit assessment for a patient planning to visit a medical facility. For example, the pre-visit service may be a service for identifying information that will be helpful during an actual medical consultation with a patient (e.g., the patient's medical history, current condition, symptoms, questions, etc.) on the basis of consultation data generated during a preliminary consultation with the patient planning to visit a medical facility and automatically scheduling an appointment or assisting with appointment scheduling on the basis of the identified information.

Here, the consultation data may be data collected during the preliminary consultation with the patient over the phone.

As an example, the consultation data may be one or more answers in the form of voice acquired from the patient when one or more preliminary questions are provided to the patient on the basis of an automatic response system (ARS).

As another example, the consultation data is data acquired through a user interface (UI) provided to the user terminal 200, and may be one or more answers in the form of text or voice acquired from the patient in response to one or more preliminary questions output through the UI.

Second, the in-visit service is a service provided using the method of automatically generating a medical chart using a generative AI model according to various embodiments of the present invention, and may be a service for automatically acquiring a medical chart during a clinical process for a patient visiting a medical facility.

An electronic medical record (EMR) system has been introduced to create an environment where medical workers may easily generate medical charts. However, this system causes medical workers to keep their hands and eyes fixed on the keyboard and monitor while generating medical charts, which results in a problem in that communication with patients is neglected.

Therefore, according to various embodiments of the present invention, the in-visit service for automatically acquiring a patient's medical chart is provided on the basis of a generative AI model. In this way, medical workers can focus on communication with patients rather than generating medical charts, and accordingly, seamless communication with patients is enabled.

Third, the post-visit service is a service provided after a patient visits a medical facility for diagnosis and treatment, and may include, but is not limited to, providing guidance on taking prescribed medications (e.g., providing a reminder at each dosage time), monitoring and recording changes in the patient's condition, analyzing test results and providing feedback, and scheduling the next appointment.

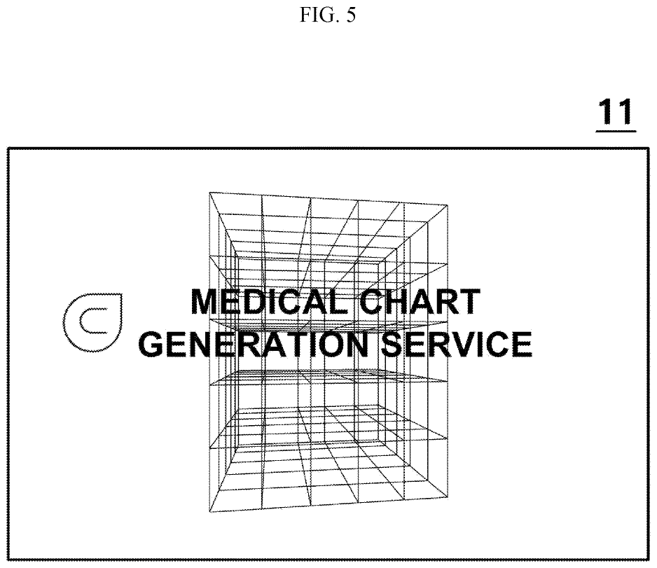

In various embodiments, the computing device 100 may be connected to the user terminal 200 via the network 400 and provide the user terminal 200 with a UI (e.g., FIGS. 5 to 8) that provides the service for automatically generating a medical chart using a generative AI model.

Here, the user terminal 200 may be any form of entity in a system with a mechanism for communication with the computing device 100. For example, the user terminal 200 may encompass a PC, a mobile terminal, a smartphone, a tablet PC, a wearable device, etc., and encompass any type of terminal that may access a wired or wireless network. Also, the user terminal 200 may encompass any computing device implemented by at least one of an agent, an application programming interface (API), and a plug-in. In addition, the user terminal 200 may include an application source and/or a client application.

Further, the network 400 may be a connective structure that enables information exchange between nodes such as a plurality of terminals and servers. For example, the network 400 may encompass a local area network (LAN), a wide area network (WAN), the Internet (the world wide web (WWW)), a wired or wireless data communication network, a telephone network, a wired or wireless television network, a controller area network (CAN), an Ethernet network, and the like.

The wireless data communication network may encompass, but is not limited to, a 3^{rd} Generation (3G) network, a 4^{th} Generation (4G) network, a 5^{th} Generation (5G) network, a 3^{rd} Generation Partnership Project (3GPP) network, a 5^{th} Generation Partnership Project (5GPP) network, a Long Term Evolution (LTE) network, a World Interoperability for Microwave Access (WiMAX) network, a Wi-Fi network, the Internet, a LAN, a wireless LAN, a WAN, a personal area network (PAN), a radio frequency (RF) network, a Bluetooth network, a near-field communication (NFC) network, a satellite broadcast network, an analog broadcast network, a digital multimedia broadcasting (DMB) network, and the like.

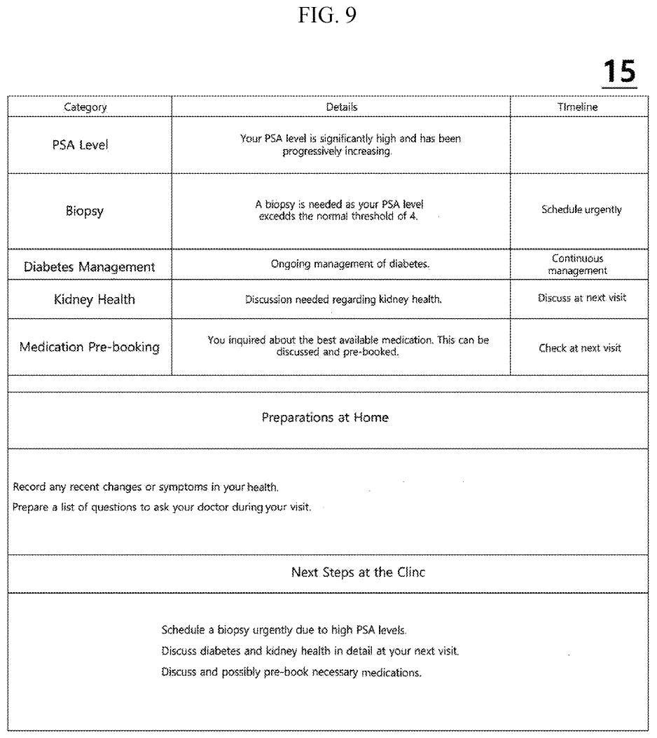

In various embodiments, the computing device 100 may automatically generate a medical document on the basis of not only a medical chart but also clinical data. For example, as shown in FIG. 9, the computing device 100 may acquire a clinical plan and a medical record on the basis of the clinical data and provide the clinical plan and medical record to the patient. Here, clinical plans and clinical notes are documents provided not to a medical expert but to patients, and thus are translated into layman's terms rather than medical jargon. Clinical plans and clinical notes may be acquired in the form of guidelines to facilitate patients' intuitive understanding, but the form of clinical plans and clinical notes is not limited thereto.

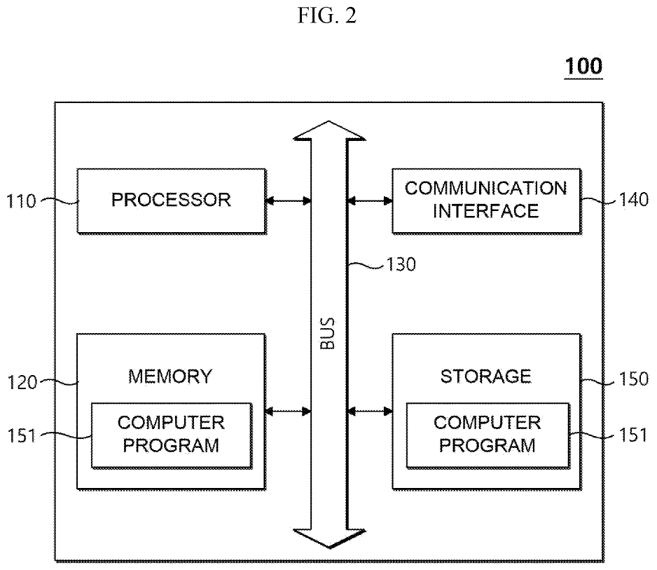

According to an embodiment, the external server 300 may be connected to the computing device 100 via the network 400 and may store and manage various information/data required for the computing device to perform the method of automatically generating a medical chart using a generative AI model or receive, store, and manage various information/data acquired when the method of automatically generating a medical chart using a generative AI model is performed by the computing device 100. For example, the external server 300 may be a storage server separately provided outside of the computing device 100 but is not limited thereto. The computing device 100 that performs the method of automatically generating a medical chart using a generative AI model will be described below with reference to FIG. 2.

FIG. 2 is a hardware configuration diagram of a computing device according to another embodiment of the present invention.

Referring to FIG. 2, in various embodiments, the computing device 100 may include at least one processor 110, a memory 120 into which a computer program 151 executed by the processor 110 is loaded, a bus 130, a communication interface 140, and a storage 150 in which the computer program 151 is stored. In FIG. 2, only components related to embodiments of the present invention are shown. Therefore, those skilled in the technical field to which the present invention pertains should appreciate that general-use components other than the components shown in FIG. 2 may be additionally included.

According to the embodiment of the present invention, the processor 110 controls overall operations of the computing device 100. The processor 110 may include a central processing unit (CPU), a micro-processor unit (MPU), a micro controller unit (MCU), a graphics processing unit (GPU), or any form of processor well known in the technical field of the present invention.

In addition, the processor 110 may perform computation for at least one application or program for executing a method according to embodiments of the present invention, and the computing device 100 may include at least one processor.

In various embodiments, the processor 110 may further include a random access memory (RAM; not shown) and a read-only memory (ROM; not shown) which temporarily and/or permanently store a signal (or data) processed in the processor 110. Also, the processor 110 may be implemented in the form of a system on chip (SoC) including at least one of a GPU, a RAM, and a ROM.

The memory 120 stores various data, commands, and/or information. The computer program 151 may be loaded from the storage 150 into the memory 120 to perform methods/operations according to various embodiments of the present invention. When the computer program 151 is loaded into the memory 120, the processor 110 may perform the methods/operations by executing one or more instructions constituting the computer program 151. The memory 120 may be implemented as a volatile memory such as a RAM, but the technical scope of the present invention is not limited thereto.

The bus 130 provides a communication function between the components of the computing device 100. The bus 130 may be implemented in various forms of buses such as an address bus, a data bus, a control bus, and the like.

The communication interface 140 supports wired or wireless Internet communication of the computing device 100. Also, the communication interface 140 may support various communication methods in addition to Internet communication. To this end, the communication interface 140 may include a communication module well known in the technical field of the present invention. In some embodiments, the communication interface 140 may be omitted.

The storage 150 may non-temporarily store the computer program 151. When the computing device 100 performs a process of automatically generating a medical chart using a generative AI model, the storage 150 may store various information required for providing the process of automatically generating a medical chart using a generative AI model.

The storage 150 may include a non-volatile memory, such as a ROM, an erasable programmable ROM (EPROM), an electrically erasable programmable ROM (EEPROM), a flash memory, etc., a hard disk, a removable disk, or any form of computer-readable recording medium well known in the technical field to which the present invention pertains.

The computer program 151 may include one or more instructions that, when loaded into the memory 120, cause the processor 110 to perform methods/operations according to various embodiments of the present invention. In other words, the processor 110 may perform the methods/operations according to various embodiments of the present invention by executing the one or more instructions.

In the embodiment, the computer program 151 may include one or more instructions for performing the method of automatically generating a medical chart using a generative AI model including a step of acquiring clinical data of a patient and a step of acquiring a medical chart corresponding to the acquired clinical data using a pretrained generative AI model.

Steps of the method or algorithm described in connection with the embodiment of the present invention may be directly implemented using hardware, implemented using software modules executed by hardware, or implemented using a combination thereof. The software modules may reside in a RAM, a ROM, an EPROM, an EEPROM, a flash memory, a hard disk, a removable disk, a compact disc (CD)-ROM, or any form of a computer-readable recording medium well known in the technical field to which the present invention pertains.

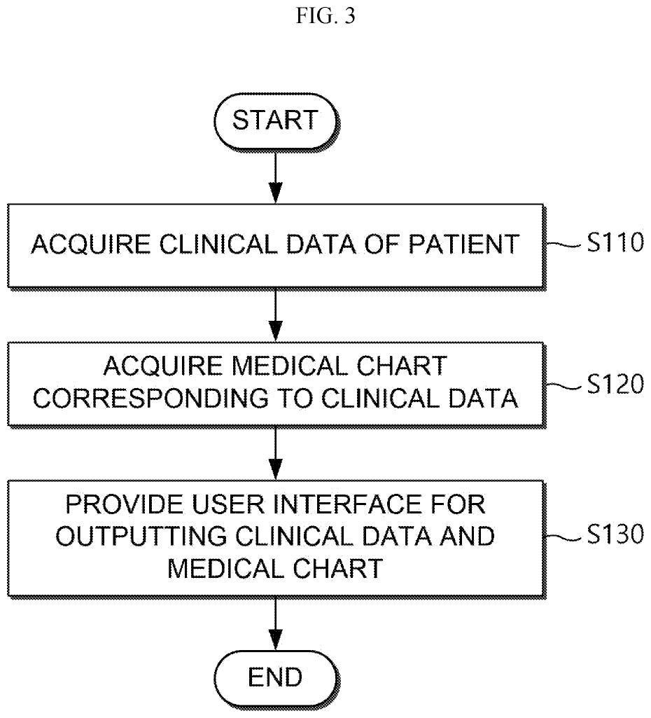

Components of the present invention may be implemented as a program (or application) and stored in a medium to be executed in combination with a computer which is hardware. Components of the present invention may be executed by software programming or software elements. Similarly, an embodiment may be implemented in a programming or scripting language, such as C, C++, Java, an assembler, etc., to include various algorithms which are embodied as combinations of data structures, processes, routines, or other programming elements. Functional aspects may be implemented using an algorithm executed by one or more processors. The method of automatically generating a medical chart using a generative AI model performed by the computing device 100 will be described below with reference to FIGS. 3 to 9.

FIG. 3 is a flowchart illustrating a method of automatically generating a medical chart using a generative AI model according to still another embodiment of the present invention.

Referring to FIG. 3, in step S110, the computing device 100 may acquire clinical data of a patient.

Here, the clinical data of the patient may be, but is not limited to, data including the patient's consultation details generated during a medical worker's consultation with the patient.

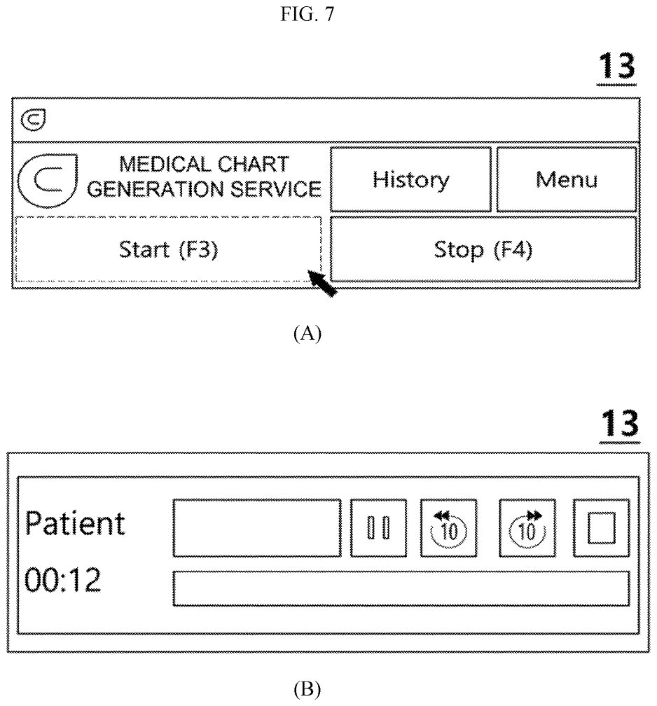

In various embodiments, the computing device 100 may acquire clinical data in the form of voice. For example, the computing device 100 may provide a UI (e.g., (A) and (B) of FIG. 7) that provides a recording function to the user terminal 200, and acquire clinical data in the form of voice that is generated by recording voice from a time point of acquiring a first user input (e.g., recording start) from the medical worker who treats the patient to a time point of acquiring a second user input (e.g., recording end). However, the clinical data is not limited thereto, and the computing device 100 may acquire various forms of clinical data in various ways.

As an example, clinical data in the form of voice that is generated and stored in advance by the medical worker recording a consultation with the patient may be uploaded to the computing device 100 via the UI.

As another example, text including the patient's consultation details may be input to the computing device 100 via the UI, or clinical data in the form of text including the consultation details may be uploaded to the computing device 100 via the UI.

As still another example, an image including manually recorded consultation details may be uploaded to the computing device 100 via the UI as clinical data in the form of an image.

It has been described that the clinical data of the patient is clinical data in the form of voice obtained by recording the patient's consultation details. However, the clinical data is not limited thereto and may encompass various forms of data such as existing medical record data of the patient, an EMR of the patient, a medical image of the patient, medical image imaging/analysis results, and the like.

In step S120, the computing device 100 may acquire a medical chart corresponding to the clinical data acquired through step S110.

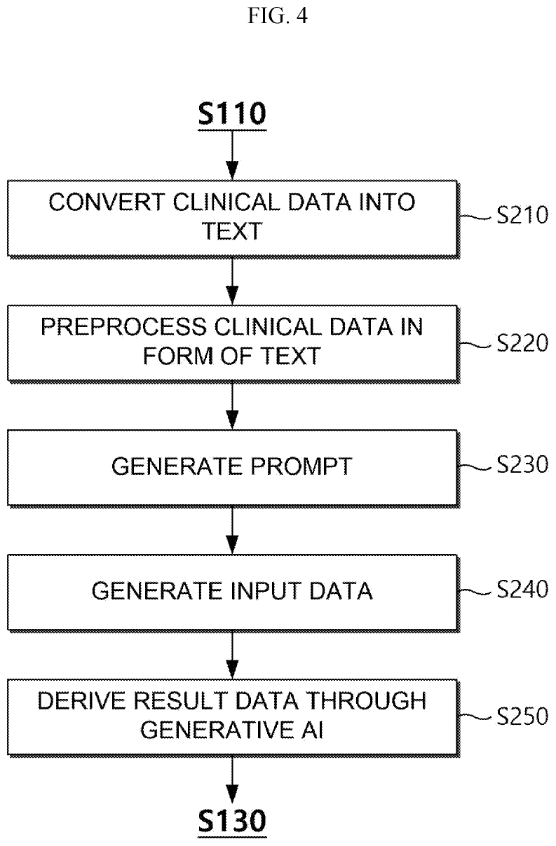

In various embodiments, the computing device 100 may acquire the medical chart corresponding to the clinical data by processing the clinical data using a generative AI model. For example, the computing device 100 may generate input data on the basis of the clinical data and input the input data into the generative AI model, thereby deriving the medical chart as result data. This will be described in further detail below with reference to FIG. 4.

FIG. 4 is a flowchart illustrating a method of acquiring a medical chart through a generative AI model according to various embodiments.

Referring to FIG. 4, in step S210, the computing device 100 may convert the clinical data in the form of voice into clinical data in the form of text.

In various embodiments, the computing device 100 may use an AI model for performing a speech to text (STT) process to convert the clinical data in the form of voice into clinical data in the form of text.

More specifically, first, the computing device 100 may remove noise included in the clinical data in the form of voice and enhance voice by preprocessing the clinical data in the form of voice.

Subsequently, the computing device 100 may extract voice characteristics by analyzing a voice signal included in the clinical data in the form of voice from which noise has been removed.

Subsequently, the computing device 100 may decompose the voice into individual words or syllables through an acoustic model among AI models for performing the STT process and match the decomposed voice to text.

In addition, the computing device 100 may understand combinations of words and the context through a linguistic model among AI models for performing the STT process and build a natural language structure.

Lastly, the computing device 100 may extract text derived through the foregoing process as clinical data in the form of text.

Here, when clinical data of the patient in the form of text is acquired through step S110 of FIG. 3, step S210 performed by the computing device 100 may be omitted, but the method is not limited thereto.

In step S220, the computing device 100 may preprocess the clinical data in the form of text converted through step S210.

Here, processing the clinical data in the form of text may be extracting only necessary information among a plurality of pieces of information included in the clinical data in the form of text or filtering out unnecessary information.

As an example, the computing device 100 may filter out unnecessary information from the clinical data using a generative AI model. For example, the computing device 100 may generate input data using the clinical data and a prompt instructing to filter out unnecessary information from the clinical data and input the generated input data into the generative AI model, thereby acquiring, as result data, unnecessary information extracted from the clinical data and the clinical data from which the unnecessary information has been filtered out.

As another example, the computing device 100 may extract information including consultation-related keywords as necessary information among the plurality of pieces of information included in the clinical data or filter out information including consultation-unrelated keywords as unnecessary information. To this end, information on consultation-related keywords and consultation-unrelated keywords may be defined and stored in advance.

As still another example, the computing device 100 may set an information filtering criterion on the basis of a type of medical chart to be acquired and extract only necessary information among the plurality of pieces of information included in the clinical data or filter out unnecessary information on the basis of the set filtering criterion.

In general, there are a variety of formats of medical charts, and the formats require different information. Accordingly, necessary information and unnecessary information may vary depending on a type of medical chart to be acquired.

For example, a subjective, objective, assessment, and plan (SOAP) medical chart format requires information on a patient's subjective account, objective test results, a diagnostic assessment, and a clinical plan but does not require information on personal details, irrelevant preceding medical records, and the like.

Also, a problem, intervention, evaluation (PIE) medical chart format requires information on an identified problem, intervention that has been taken, and the evaluation of the intervention's effectiveness but does not require any observation or data not directly related to the problem.

Further, a focus, objective, charting, action, summary (FOCUS) medical chart format requires information on focus on a specific issue, objective data, measures, and a summary but does not require details unrelated to the primary issue.

In other words, each medical chart format has different necessary information and different unnecessary information. In consideration of this, the computing device 100 may define which information is necessary or which information is unnecessary as the information filtering criterion on the basis of a type of medical chart to be acquired and enable the acquisition of an accurate medical chart by extracting only necessary information or filtering out unnecessary information on the basis of the information filtering criterion.

In step S230, the computing device 100 may generate a prompt for acquiring a medical chart.

In various embodiments, the computing device 100 may generate a prompt with a preset structure.

Here, the preset structure may include the type of medical chart to be acquired and a command to generate that type of medical chart.

For example, when the type of medical chart to be acquired is a SOAP medical chart, the prompt with the preset structure may have a structure that induces derivation of information in the form of a SOAP medical chart (e.g., “Please summarize the conversation between the patient and the doctor and reorganize the conversation into the SOAP medical chart format”) such that a SOAP medical chart may be acquired on the basis of clinical data.

In step S240, the computing device 100 may generate input data that includes the clinical data in the form of text converted through step S210 or the clinical data in the form of text preprocessed through step S220 and the prompt generated through step S230.

In step S250, the computing device 100 may acquire a medical chart corresponding to the clinical data by inputting the input data generated through step S240 into the generative AI model.

In various embodiments, the computing device 100 may select a suitable medical chart format for the clinical data and acquire a medical chart in accordance with the selected medical chart format. For example, the computing device 100 may extract a plurality of keywords from the clinical data, select any one of a plurality of preset medical chart formats on the basis of the plurality of keywords, and acquire a medical chart by processing the clinical data in accordance with the selected medical chart format.

In various embodiments, the computing device 100 may extract a medical department and disease information by analyzing the clinical data, select any one of the plurality of preset medical chart formats in accordance with the extracted clinical department and disease, and acquire a medical chart by processing the clinical data in accordance with the selected medical chart format.

In various embodiments, the computing device 100 may extract keywords corresponding to each of a plurality of items included in the preset medical chart format from the clinical data, acquire information corresponding to each of the plurality of items on the basis of the keywords, and acquire a medical chart in a preset medical chart format by placing the acquired information in areas corresponding to each of the plurality of items.

Here, when a keyword corresponding to any one of the plurality of items is not extracted, the computing device 100 may acquire information corresponding to the single item on the basis of keywords extracted in accordance with the items other than the single item.

As an example, in the case of trying to acquire a SOAP medical chart, the computing device 100 may acquire information corresponding to each of a subjective(S) item, an objective (O) item, an assessment (A) item, and a plan (P) item on the basis of the keywords extracted from the medical data. When no keyword corresponding to the plan (P) item (e.g., a keyword related to a treatment and follow-up plan for the patient) is extracted, the computing device 100 may predict a treatment and follow-up plan for the patient on the basis of keywords extracted in accordance with the subjective(S) item, the objective (O) item, and the assessment (A) item and acquire the content of the plan (P) item on the basis of the predicted treatment and follow-up plan.

As another example, when no keyword corresponding to the plan (P) item (e.g., a keyword related to a treatment and follow-up plan for the patient) is extracted, the computing device 100 may derive a treatment and follow-up plan for the patient on the basis of a pretrained consultation, diagnosis, and prescription model (e.g., a model trained on data such as papers/clinical notes, etc.) and acquire information on the derived treatment and follow-up plan as information corresponding to the plan (P) item.

As still another example, when no keyword corresponding to the plan (P) item (e.g., a keyword related to a treatment and follow-up plan for the patient) is extracted, the computing device 100 may acquire, as information corresponding to the plan (P) item, prescription information preset through a rule-based model.

Here, the preset prescription information may be, but is not limited to, prescription information based on orders agreed upon and coordinated in advance among medical workers such as prescription information preset in accordance with a diagnostic name, prescription information preset in accordance with a case where no keyword corresponds to the plan (P) item, or the like.

In various embodiments, when the computing device 100 is requested by a user to acquire a medical chart in accordance with a plurality of different medical chart formats, the computing device 100 may generate a prompt instructing to generate a medical chart in accordance with the plurality of different medical chart formats and input the prompt into the generative AI model, thereby acquiring a plurality of medical charts corresponding to each of the plurality of different medical chart formats.

In various embodiments, when no type of medical chart to be acquired is specified by the user, the computing device 100 may determine a type of medical chart that is acquirable on the basis of the keywords extracted by analyzing the clinical data, and acquire a medical chart in accordance with the determined type of medical chart format. For example, when a SOAP medical chart, a PIE medical chart, and a FOCUS medical chart are acquirable on the basis of the clinical data, the computing device 100 may acquire each of the SOAP medical chart, the PIE medical chart, and the FOCUS medical chart using the clinical data, but acquirable medical charts are not limited thereto.

In various embodiments, the computing device 100 may verify the medical chart acquired through the foregoing process.

As an example, the computing device 100 may generate clinical data for verification using the medical chart acquired through the foregoing process, calculate a similarity between the original clinical data used for acquiring the medical chart and the clinical data for verification, and determine suitability of the medical chart depending on whether the calculated similarity is a threshold or more.

As another example, the computing device 100 may provide the user with the medical chart acquired through the foregoing process, and when feedback indicating that the medical chart is unsuitable is received from the user, may reacquire a medical chart on the basis of the clinical data.

Here, the computing device 100 may receive from the user a reason that the medical chart is unsuitable in a multiple-choice or open-ended format and add the reason received from the user to the prompt such that a medical chart may be reacquired to reflect the reason that the medical chart is unsuitable.

In various embodiments, when clinical data in the form of voice and clinical data in the form of an image (e.g., an image obtained by capturing consultation details manually input through the UI output on the user terminal 200 using a touch pen, etc.) are acquired together as clinical data of the patient, the computing device 100 may acquire a medical chart on the basis of the clinical data in the form of voice and store the medical chart and the clinical data in the form of an image in association with each other.

Referring back to FIG. 3, in step S130, the computing device 100 may provide a UI for outputting the clinical data acquired through step S110 and the medical chart acquired through step S120.

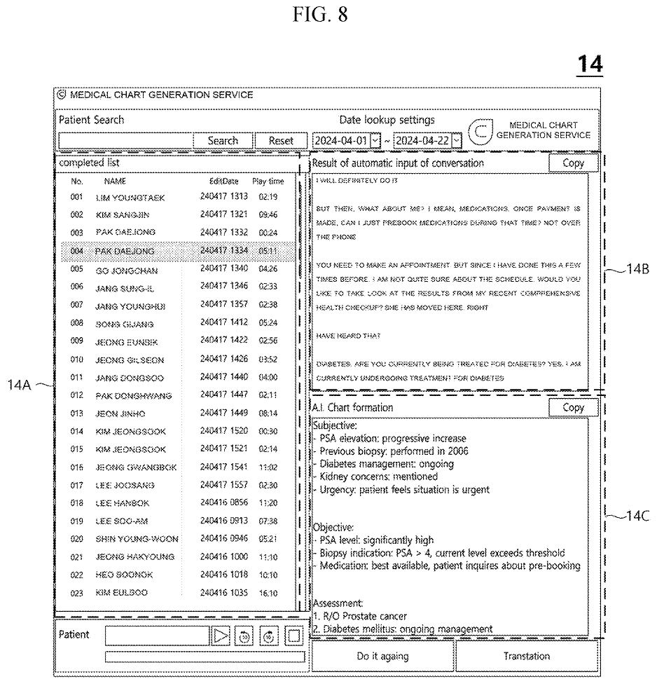

As shown in FIG. 8, the UI may include, but is not limited to, a first region where clinical data is output and a second region where a medical chart is output.

In various embodiments, when a plurality of different kinds of medical charts are acquired in accordance with the clinical data, the computing device 100 may separately output the plurality of medical charts in different regions of the UI. For example, the computing device 100 may generate a UI including a plurality of tabs which are separately output in each of a plurality of different medical charts, and may provide the UI to the user terminal 200, but a UI generated and provided by the computing device is not limited thereto.

In various embodiments, the computing device 100 may output specific clinical data together with a specific medical chart acquired on the basis of the specific clinical data via the UI. However, when one or more pieces of unnecessary information are filtered out from the specific clinical data during a process of generating the specific medical chart, the computing device 100 may aggregate the one or more pieces of unnecessary information and provide the unnecessary information together with the specific medical chart. For example, the computing device 100 may output the clinical data in the first region of the UI, output the medical chart in an upper part of the second region of the UI, and output filtered unnecessary information in a lower part of the second region of the UI.

In this case, the computing device 100 may simultaneously output the unnecessary information and information on a reason that the unnecessary information has been filtered out from the clinical data (e.g., irrelevant to medical consultations, etc.), but the present invention is not limited thereto.

In other words, the computing device 100 generates a report including clinical data, a medical chart, and unnecessary information filtered out from the clinical data and provides a UI for outputting the generated report, enabling a user to check the clinical data, the medical chart, and the unnecessary information all on a single screen, thereby enhancing the efficiency of medical consultations.

The method of automatically generating a medical chart using a generative AI model has been described with reference to flowcharts illustrated in the drawings. For simplicity of description, the method of generating a medical chart using a generative AI model has been shown and described as a series of blocks, but the present invention is not limited to the order of blocks. Some blocks may be performed simultaneously or in a different order from that illustrated and described herein. In addition, the method may be performed with added new blocks which are not illustrated in this specification or the drawings or some blocks removed or changed. The UI provided by the computing device 100 will be described below with reference to FIGS. 5 to 9.

FIGS. 5 to 9 are diagrams illustrating UIs applicable to various embodiments.

First, referring to FIG. 5, when a service execution request (e.g., execution of an application for providing an automatic medical chart generation service) is acquired from a user via the user terminal 200, the computing device 100 may provide a first UI 11 to the user terminal 200.

The first UI 11 may be a UI that outputs a start screen of the automatic medical chart generation service, but is not limited thereto.

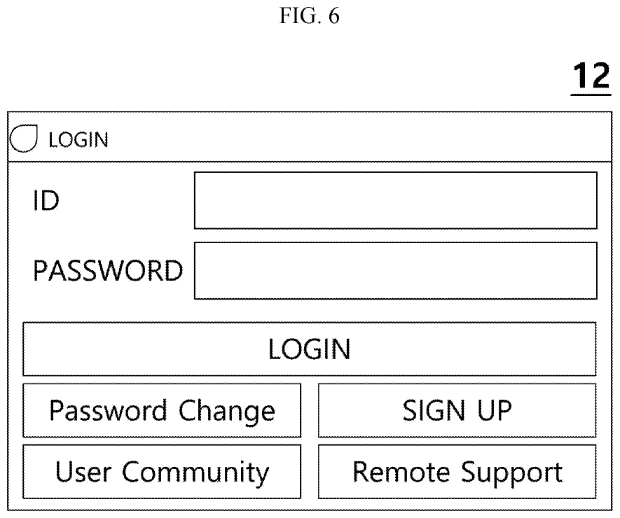

Subsequently, referring to FIG. 6, when a certain time elapses after the first UI 11 is provided to the user terminal 200 or an input of touching at least a part of the first UI 11 is acquired from the user, the computing device 100 may provide a second UI 12 to the user terminal 200.

Here, the second UI 12 may be a UI that outputs a login screen for receiving the user's account information required for using the automatic medical chart generation service. For example, the second UI 12 may include, but is not limited to, a user account information input field (ID), a password input field (PASSWORD), a login button (LOGIN), a password change button (Password Change), a membership signup button (SIGN UP), a user community entry button (User Community), and a remote support button (Remote Support).

Subsequently, referring to (A) and (B) of FIG. 7, when a clinical data upload request is acquired from a user who wants to acquire a medical chart, the computing device 100 may provide a third UI 13 to the user terminal 200.

Here, the third UI 13 may be a UI that provides a recording function. For example, the third UI may include a recording start button (Start), a recording end button (Stop), a recording history button (History), and a menu button (Menu).

For example, as shown in (A) of FIG. 7, the third UI 13 may include the recording start button (Start). When a first user input of selecting the recording start button is acquired from the user, the computing device 100 may activate a recording function as shown in (B) of FIG. 7 to record voice input via a voice input device (e.g., a microphone).

Also, as shown in (A) of FIG. 7, the third UI 13 may include the recording end button (Stop). When a second user input of selecting the recording end button is acquired from the user with the recording function activated, the computing device 100 may stop the recording function and generate clinical data in the form of voice by recording voice from a time point where the first user input is acquired to a time point where the second user input is acquired.

Subsequently, referring to FIG. 8, when the clinical data is acquired from the user, the computing device 100 may provide a fourth UI 14 to the user terminal 200.

Here, the fourth UI 14 may be a UI that outputs a medical chart acquired in accordance with the clinical data acquired from the user. For example, the fourth UI 14 may include a clinical data list output region, a first region, a second region, a medical chart conversion button (Translation), and a reconversion button (Do it again).

The clinical data list output region 14A may be a region where a clinical data list in which the plurality of pieces of clinical data acquired from the user are arranged under a preset condition (e.g., in order of clinical data upload, alphabetical order by patient name, in order of medical chart acquisition time, etc.) is output.

The first region 14B may be a region where the clinical data is output. For example, when specific clinical data is selected by the user from among the plurality of pieces of clinical data output in the clinical data list output region, the computing device 100 may output text corresponding to the specific clinical data in the first region 14B.

The second region 14C may be a region where the medical chart is output. For example, when specific clinical data is selected by the user from among the plurality of pieces of clinical data output in the clinical data list output region, the computing device 100 may output a medical chart corresponding to the specific clinical data in the second region 14C.

Here, when there is a pre-acquired medical chart corresponding to the specific clinical data, the computing device 100 may output the pre-acquired medical chart corresponding to the specific clinical data in the second region 14C. On the other hand, when there is no pre-acquired medical chart corresponding to the specific clinical data, the computing device 100 may guide the user to obtain a medical chart for the specific clinical data by selecting the medical chart conversion button (Translation).

Further, when a user input of selecting the reconversion button (Do it again) is acquired from the user, the computing device 100 may reacquire a medical chart corresponding to the clinical data output in the first region 14B and output the regenerated medical chart in the second region 14C.

However, the present invention is not limited thereto. When at least some content of the specific clinical data output in the first region 14B is corrected by the user, the computing device 100 may automatically reacquire a medical chart in accordance with the corrected content and output the reacquired medical chart in the second region 14C.

When a plurality of medical charts are acquired for the specific clinical data output in the first region 14B, the computing device 100 may separate the plurality of medical charts to different tabs and output each of the medical charts in the second region 14C included in each of the tabs.

In various embodiments, when specific information from the specific clinical data output in the first region 14B is determined as unnecessary information, the computing device 100 may classify the specific information as consultation-unrelated information and display that the specific information is classified and filtered out as unnecessary information in the first region 14B (e.g., strikethrough, highlighting, inputting text clarifying unnecessary information, etc.).

Lastly, when a request for generating a clinical note to be provided to a patient is acquired from the user, the computing device 100 may provide a fifth UI 15.

The fifth UI 15 may be a UI that outputs the clinical note written on the basis of the clinical data.

Here, clinical plans and clinical notes are documents provided not to a medical expert but to patients and thus are translated into layman's terms rather than medical jargon. Clinical plans and clinical notes may be acquired in the form of guidelines to facilitate patients' intuitive understanding and may be output in the fifth UI 15.

In addition, the computing device 100 may provide UIs with various functions such as a UI with pen and drawing functions, a UI for receiving clinical data in the form of text or images such that a user may manually record consultation details of a patient with convenience during a medical consultation with the patient.

Although embodiments of the present invention have been described with reference to the accompanying drawings, those of ordinary skill in the art should appreciate that the present invention can be implemented in other specific forms without changing the technical spirit of essential characteristics of the present invention. Therefore, it is to be understood that the embodiments described above are illustrative in all aspects and not limiting.

1. A method of automatically generating a medical chart using a generative artificial intelligence (AI) model, which is performed by a computing device, the method comprising:
acquiring clinical data of a patient; and
acquiring a medical chart corresponding to the acquired clinical data using a pretrained generative AI model.

2. The method of claim 1, wherein the acquired clinical data is data including information on a medical consultation with the patient and is data in the form of voice generated by recording voice from a time point where a first user input is acquired from a medical worker who treats the patient to a time point where a second user input is acquired.

3. The method of claim 1, wherein the acquiring of the medical chart comprises:
generating input data on the basis of the acquired clinical data; and
acquiring a medical chart corresponding to the acquired clinical data as resulting data by inputting the generated input data into the pretrained generative AI model.

4. The method of claim 3, wherein the acquired clinical data includes clinical data in the form of voice obtained by recording a medical consultation with the patient, and
the generating of the input data comprises:
converting the clinical data in the form of voice into clinical data in the form of text;
generating a prompt including a command to indicate a type of medical chart to be acquired and generate a medical chart; and
generating input data including the converted clinical data in the form of text and the generated prompt.

5. The method of claim 1, wherein the acquiring of the medical chart comprises:
preprocessing the acquired clinical data; and
acquiring a medical chart using the preprocessed clinical data.

6. The method of claim 5, wherein the preprocessing of the acquired clinical data comprises extracting information including a consultation-related keyword among a plurality of pieces of information included in the acquired clinical data as required information or filtering out information including a consultation-unrelated keyword as unrequired information.

7. The method of claim 5, wherein the preprocessing of the acquired clinical data comprises:
setting an information filtering criterion on the basis of a type of medical chart to be acquired; and
extracting only necessary information among a plurality of pieces of information included in the acquired clinical data or filtering out unnecessary information on the basis of the set information filtering criterion.

8. The method of claim 1, wherein the acquiring of the medical chart comprises:
analyzing the acquired clinical data to extract a plurality of keywords;
selecting any one from a plurality of preset medical chart formats on the basis of the plurality of extracted keywords; and
acquiring a medical chart by processing the acquired clinical data in accordance with the selected medical chart format.

9. The method of claim 1, wherein the acquiring of the medical chart comprises extracting keywords corresponding to each of a plurality of items included in a preset medical chart format from the acquired clinical data and acquiring information corresponding to each of the plurality of items on the basis of the extracted keywords,
wherein, when no keyword corresponding to any one of the plurality of items is extracted, acquiring information corresponding to the item on the basis of keywords extracted in accordance with the items other than the item.

10. The method of claim 9, wherein the preset medical chart format is a subjective, objective, assessment, and plan (SOAP) medical chart format including a subjective item where information on a patient's complaint is recorded, an objective item where factual information collected during a consultation process is recorded by a medical worker, an assessment item where assessment information generated by a medical worker assessing the patient's current condition is recorded, and a plan item where treatment and follow-up plan information for the patient is recorded.

11. The method of claim 1, further comprising providing a user interface (UI) configured to output the acquired clinical data and the acquired medical chart,
wherein the provided UI includes a first region where the acquired clinical data is output and a second region where the acquired medical chart is output.

12. The method of claim 11, wherein the providing of the UI comprises, when a plurality of different kinds of medical charts are acquired in accordance with the acquired clinical data, separately outputting the plurality of acquired medical charts in different regions of the provided UI.

13. A computing device for performing a method of automatically generating a medical chart using a generative artificial intelligence (AI) model, the computing device comprising:
a processor;
a network interface;
a memory; and
a computer program loaded into the memory and executed by the processor,
wherein the computer program comprises:
an instruction to acquire clinical data of a patient; and
an instruction to acquire a medical chart corresponding to the acquired clinical data using a pretrained generative AI model.

14. A computer program stored in a computer-readable recording medium to perform, in combination with a computing device, a method of automatically generating a medical chart using a generative artificial intelligence (AI) model, wherein the method comprises:
acquiring clinical data of a patient; and
acquiring a medical chart corresponding to the acquired clinical data using a pretrained generative AI model.
